(19) United States
(12) Patent Application Publication
(10) Pub. No.: US 2023/0410400 A1
(43) Pub. Date: Dec. 21, 2023

(54) BODY SHAPE MANAGEMENT SYSTEM, ITS CONTROL METHOD, AND CONTROL PROGRAM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Masahito MATSUSHIMA, Edogawa-ku (JP); Hironori ITO, Edogawa-ku (JP); Ryota NAKABAYASHI, Nagoya-shi (JP); Maxwell FORREST, Kawasaki-shi (JP); Shotaro YASUDA, Taito-ku (JP)

(73) Assignee: TOYOTA JIDOSHA KABUSHIKI KAISHA, Toyota-shi (JP)

(21) Appl. No.: 18/337,010

(22) Filed: Jun. 18, 2023

(30) Foreign Application Priority Data
Jun. 20, 2022 (JP) 2022-099188

(51) Int. Cl. G06T 13/40

(52) U.S. Cl. CPC G06T 13/40

(57) ABSTRACT

A body shape management system includes: an avatar output unit configured to post an avatar representing a future body shape of a first user in a chat room in association with a message posted by the first user in the chat room, the future body shape of the first user being his/her body shape expected after a lapse of a set period; an extraction unit configured to extract a message containing a predetermined content from a message posted for the first user by a second user different from the first user; and an avatar adjustment unit configured to change the future body shape of the avatar of the first user according to the content of the message extracted by the extraction unit.

### CROSS REFERENCE TO RELATED APPLICATIONS

This application is based upon and claims the benefit of priority from Japanese patent application No. 2022-099188, filed on Jun. 20, 2022, the disclosure of which is incorporated herein in its entirety by reference.

### BACKGROUND

The present disclosure relates to a body shape management system, its control method, and a control program.

Japanese Unexamined Patent Application Publication No. 2007-310632 discloses an avatar health and body shape prediction system capable of displaying an avatar of a user and predicting his/her own body shape and health condition that he/she will have after a lapse of an arbitrary period.

### SUMMARY

It has been desired to encourage users to improve their body shapes.

The present disclosure has been made in view of the above-described circumstances, and an object thereof is to provide a body shape management system, its control method, and a control program suitable to encourage users to improve their body shapes.

A body shape management system according to the present disclosure includes: an avatar output unit configured to post an avatar representing a future body shape of a first user in a chat room in association with a message posted by the first user in the chat room, the future body shape of the first user being his/her body shape expected after a lapse of a set period; an extraction unit configured to extract a message containing a predetermined content from a message posted for the first user by a second user different from the first user; and an avatar adjustment unit configured to change the future body shape of the avatar of the first user according to the content of the message extracted by the extraction unit. The above-described body shape management system changes a future body shape of an avatar of a user that is posted together with a message from that user in a chat room in response to an encouraging message from other users in such a manner that, for example, the future body shape of the avatar gets closer to a desired body shape of the user. In this way, it is possible to make the user be aware that, for example, he/she is being cheered up by other users, and thereby to encourage the user to improve his/her body shape.

When one of a message containing a content complimenting an effort of the first user to achieve the desired body shape, a message containing a content complimenting the first user that his/her body shape has gotten closer to his/her desired body shape, and a message containing a content cheering the first user up so that his/her body shape gets closer to his/her desired body shape is extracted from among messages containing the predetermined content posted for the first user by the extraction unit, the avatar adjustment unit may change the future body shape of the avatar of the first user so that it gets closer to the desired body shape.

The body shape management system may further include an acquisition unit configured to acquire information about an actual body shape of the first user, in which the avatar output unit may post an avatar representing a future body shape corresponding to the actual body shape of the first user in the chat room.

The body shape management system may further include a predicted body shape calculation unit configured to calculate a predicted body shape of the first user according to at least a schedule of the first user stored in a database, the predicted body shape of the first user being a body shape that it is predicted the first user will have after a lapse of a set period, in which the avatar output unit may post an avatar representing a future body shape corresponding to the predicted body shape of the first user calculated by the predicted body shape calculation unit in the chat room.

The body shape management system may further include a notification unit configured to, when it is determined that the actual body shape of the first user has gotten closer to his/her desired body shape owing to the message extracted by the extraction unit, notify the first user that his/her body shape has gotten closer to his/her desired body shape.

When it is determined that the actual body shape of the first user has gotten closer to his/her desired body shape owing to the message extracted by the extraction unit, the notification unit may post a message indicating that his/her body shape has gotten closer to his/her desired body shape in the chat room.

When it is determined that the actual body shape of the first user has gotten closer to his/her desired body shape owing to the message extracted by the extraction unit, the avatar adjustment unit may increase, after this extracted message containing the predetermined content, a degree of change in the future body shape of the avatar of the first user that occurs in response to a subsequent message containing the predetermined content posted by the second user, who has posted the extracted message.

When it is determined that the actual body shape of the first user has gotten closer to his/her desired body shape owing to the message extracted by the extraction unit, the avatar output unit may attach a mark to an avatar of the second user, who has posted the extracted message, or in a vicinity of the avatar of the second user, the mark indicating a contributing result by which the actual body shape of the first user has gotten closer to his/her desired body shape.

The avatar output unit may change at least one of a color and a shape of the avatar of the second user, who has posted the message extracted by the extraction unit, according to the contributing result by which the actual body shape of the first user has gotten closer to his/her desired body shape.

The set period may be changeable.

A method for controlling a body shape management system according to the present disclosure includes: posting an avatar representing a future body shape of a first user in a chat room in association with a message posted by the first user in the chat room, the future body shape of the first user being his/her body shape expected after a lapse of a set period; extracting a message containing a predetermined content from a message posted for the first user by a second user different from the first user; and changing the future body shape of the avatar of the first user according to the content of the extracted message. In the above-described control method for a body shape management system, a future body shape of an avatar of a user that is posted together with a message from that user in a chat room is changed in response to an encouraging message from other users in such a manner that, for example, the future body shape of the avatar gets closer to a desired body shape of the user. In this way, it is possible to make the user be aware that, for example, he/she is being cheered up by other users, and thereby to encourage the user to improve his/her body shape.

A control program according to the present disclosure causes a computer to perform: a process for posting an avatar representing a future body shape of a first user in a chat room in association with a message posted by the first user in the chat room, the future body shape of the first user being his/her body shape expected after a lapse of a set period; a process for extracting a message containing a predetermined content from a message posted for the first user by a second user different from the first user; and a process for changing the future body shape of the avatar of the first user according to the content of the extracted message. By the above-described control program, a future body shape of an avatar of a user that is posted together with a message from that user in a chat room is changed in response to an encouraging message from other users in such a manner that, for example, the future body shape of the avatar gets closer to a desired body shape of the user. In this way, it is possible to make the user be aware that, for example, he/she is being cheered up by other users, and thereby to encourage the user to improve his/her body shape.

According to the present disclosure, it is possible to provide a body shape management system, its control method, and a control program suitable to encourage users to improve their body shapes.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a body shape management system according to a first embodiment;

FIG. 2 is a flowchart showing operations performed by the body shape management system according to the first embodiment;

FIG. 3 shows an example of a chat room displayed on a monitor of a user terminal; and

FIG. 4 shows an example of a chat room displayed on a monitor of a user terminal.

### DESCRIPTION OF EMBODIMENTS

The present disclosure will be described hereinafter through embodiments of the disclosure, but the invention according to the claims is not limited to the below-shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### First Embodiment

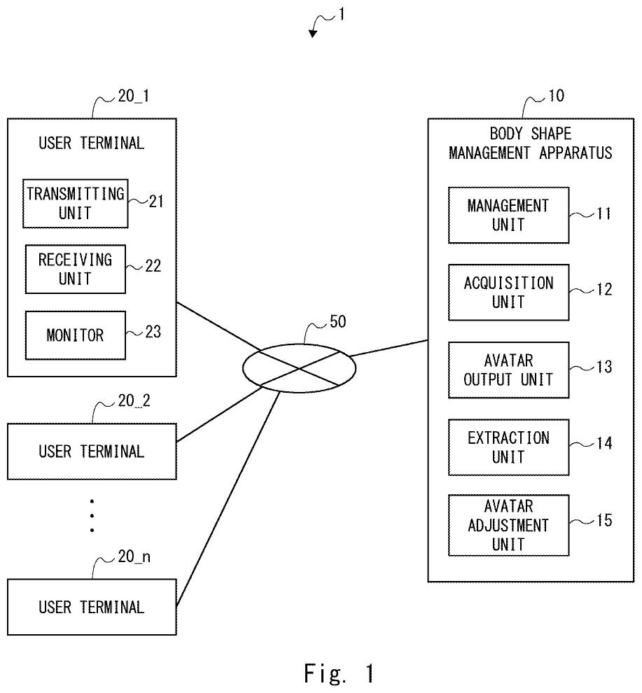

FIG. 1 is a block diagram showing an example of a configuration of a body shape management system 1 according to the first embodiment. The body shape management system 1 changes a future body shape of an avatar of a user that is posted together with a message from that user in a chat room in response to an encouraging message from other users in such a manner that, for example, the future body shape of the avatar gets closer to a desired body shape of the user. In this way, the body shape management system 1 can make the user be aware that, for example, he/she is being cheered up by other users, and thereby can encourage the user to improve his/her body shape. Details of the body shape management system 1 or the like will be described hereinafter.

As shown in FIG. 1, the body shape management system 1 includes a body shape management apparatus 10, n user terminals 20 (n is an integer equal to or greater than 2), and a network 50. The body shape management apparatus 10 can also be referred to as a body shape management system. In the following description, the n user terminals 20 are also referred to as user terminals 20_1 to 20_n, respectively. The body shape management apparatus 10 and the user terminals 20_1 to 20_n are configured so that they can communicate with each other through the network 50.

Each of the user terminals 20_1 to 20_n is a terminal capable of performing wireless communication, owned by or temporarily assigned to a user, such as a PC (Personal Computer) terminal, a mobile terminal such as a smartphone or a tablet-type terminal, or a dedicated communication terminal provided for the body shape management system. In this embodiment, an example case where the user terminals 20_1 to 20_n are mobile terminals such as smartphones owned by users U1 to Un, respectively, will be described.

The user terminal 20_1 includes at least a transmitting unit 21, a receiving unit 22, and a monitor 23. For example, the user U1 enters a message to be posted in a chat room into the user terminal 20_1 by operating an operation screen or the like of the user terminal 20_1. The transmitting unit 21 receives the message entered by the operation performed by the user U1, and transmits the received message to the body shape management apparatus 10 through the network 50. The receiving unit 22 receives information such as a message posted in a chat room managed by the body shape management apparatus 10 through the network 50. The monitor 23 displays the message posted in the chat room received by the receiving unit 22. Note that the transmitting unit 21 may receive not only the message to be posted in the chat room, but also information about the actual body shape of the user U1 and/or information about a desired body shape that the user U1 wants to have after a lapse of a set period, and transmit the received information items to the body shape management apparatus 10 through the network 50.

Note that the information about the actual body shape of the user U1 means the apparent body shape of the user U1 at the present time (at the time when the information is entered). The information about the actual body shape of the user U1 may be directly entered by the user U1, or may be estimated by the body shape management apparatus 10 or the like based on information about, for example, the age, gender, height, and weight of the user U1 at the present time.

Further, the information about the desired body shape that the user U1 wants to have after the lapse of the set period is the apparent body shape that the user U1 wants to have after the lapse of the set period. The set period can be arbitrarily set by the user U1, and can be set to, for example, one month, three months, or one year. The information about the desired body shape that the user U1 wants to have after the lapse of the set period may be directly entered by the user U1, or may be estimated by the body shape management apparatus 10 or the like based on information about, for example, a target weight and/or a target body fat percentage of the user U1 that he/she wants to have after the lapse of the set period.

The configuration of each of the user terminals 20_2 to 20_n is similar to that of the user terminal 20_1 except that its owner is different, and therefore descriptions thereof are omitted.

The body shape management apparatus 10 manages the chat room. Note that the body shape management apparatus 10 posts, in the chat room, an avatar representing a future body shape, i.e., a body shape expected after a lapse of a set period, of a user who has posted a message in the chat room in association with the posted message.

Specifically, the body shape management apparatus 10 includes at least a management unit 11, an acquisition unit 12, an avatar output unit 13, an extraction unit 14, and an avatar adjustment unit 15.

The management unit 11 manages the posting of messages in the chat room by a plurality of users who are permitted to use the chat room. Note that the management unit 11 may be provided as an apparatus separate from the body shape management apparatus 10.

In this embodiment, an example case where, for example, the management unit 11 manages the posting of messages in the chat room by users U1 to Un who are permitted to use the chat room will be described.

The acquisition unit 12 acquires a message from a user Ui to be posted in the chat room, transmitted from an arbitrary user terminal 20_i (i is an integer from 1 to n) among the user terminals 20_1 to 20_n. Note that the user Ui is any user among the users U1 to Un, and a user who owns the user terminal 20_i. The message from the user Ui acquired by the acquisition unit 12 is posted in the chat room by the management unit 11. Note that the acquisition unit 12 may further acquire, for example, information about the actual body shape of the user Ui and/or information about a desired body shape that the user Ui wants to have after a lapse of a set period, transmitted from the user terminal 20_i.

The avatar output unit 13 posts, in the chat room, an avatar representing a future body shape, i.e., a body shape expected after a lapse of a set period, of the user Ui who has posted a message in the chat room in association with the message posted by the user Ui. For example, when a message is posted by the user U1 in the chat room, the avatar output unit 13 posts an avatar representing a future body shape, i.e., a body shape expected after a lapse of a set period, of the user U1 in the chat room in association with the message posted by the user U1.

Note that in an initial state, the avatar representing the future body shape of the user Ui expected after the lapse of the set period represents a body shape of the user Ui before he/she has his/her desired body shape, and can be edited (i.e., modified) as desired through an operation or the like performed on the user terminal 20_i by the user Ui.

Note that when information about the actual body shape of the user Ui is acquired by the acquisition unit 12, the avatar output unit 13 may generate an avatar representing a future body shape, i.e., a body shape expected after a lapse of a set period, of the user Ui by referring to the actual body shape of the user Ui, and post the generated avatar in the chat room. That is, the avatar output unit 13 may generate an avatar representing an actual body shape of the user Ui or an avatar corresponding thereto, and post the generated the avatar in the chat room.

The extraction unit 14 extracts a message containing a predetermined content from among messages posted in the chat room. For example, the extraction unit 14 extracts a message containing a term or an expression indicating a predetermined content stored in a database (not shown) from among messages posted in the chat room. Note that in the case of messages posted for the user U1 by the user U2, the message containing the predetermined content includes one of a message containing a content complimenting an effort of the user U1 to achieve the desired body shape, a message containing a content complimenting the user U1 that his/her actual body shape has gotten closer to his/her desired body shape, and a message containing a content cheering the user U1 up so that his/her actual body shape gets closer to his/her desired body shape.

When a message containing a predetermined content is extracted by the extraction unit 14, the avatar adjustment unit 15 changes the future body shape of the avatar of the user Ui, who has received the message, so that, for example, the body shape of the avatar gets closer to the desired body shape. Note that the avatar adjustment unit 15 may change the degree of change in the future body shape of the avatar of the user Ui, who has received the message, according to the content of the message extracted by the extraction unit 14.

Each of the user terminals 20_1 to 20_n can acquire (receive) information in the chat room and display the acquired information on a monitor by accessing the chat room through the network 50 through an operation performed by a respective one of the users U1 to Un. For example, as the user U1 watches that the future body shape of his/her avatar in the chat room displayed on the monitor of the user terminal 20_1 changes, the user U1 becomes aware that he/she is being cheered up by other users and/or receives attention from other users, so that it is possible to encourage the user U1 to improve his/her body shape.

As described above, the body shape management system 1 according to this embodiment changes the future body shape of an avatar of a user that is posted together with a message from that user in the chat room in response to an encouraging message from other users in such a manner that, for example, the future body shape of the avatar gets closer to the desired body shape of the user. In this way, the body shape management system 1 can make the user be aware that, for example, he/she is being cheered up by other users, and thereby can encourage the user to improve his/her body shape.

Note that the body shape management system 1 may further include a predicted body shape calculation unit that calculates, for each of the users U1 to Un, a predicted body shape of that user, i.e., a body shape that it is predicted the user will have after a lapse of a set period, according to a schedule of that user stored in a database (not shown). In this case, the avatar output unit 13 may generate, for each of the users U1 to Un, an avatar representing a future body shape, i.e., a body shape expected after a lapse of a set period, of that user by referring to a predicted body shape of that user, i.e., a body shape that it is predicted the user will have after the lapse of the set period, calculated by the predicted body shape calculation unit, and post the generated avatar in the chat room. That is, the avatar output unit 13 may generate, for each of the users U1 to Un, an avatar corresponding to a predicted body shape of that user, i.e., a body shape that it is predicted the user will have after a lapse of a set period, and post the generated avatar in the chat room.

Further, the body shape management system 1 may further include a notification unit that, when it is determined that the actual body shape of the user, who has received the message extracted by the extraction unit 14, has gotten closer to his/her desired body shape owing to that message, notifies the user that his/her body shape has gotten closer to his/her desired body shape. The content of this notification by the notification unit is transferred to a respective one of the user terminals 20_1 to 20_n through the network 50, and is displayed on the monitor or output in the form of voice or the like. Alternatively, the content of this notification by the notification unit may be posted in the chat room. In this way, users other than the user who has received the cheering message may have senses of rivalry, so that it is possible to encourage them to improve their body shapes. Note that the determination as to whether or not the body shape of the user Ui has gotten closer to his/her desired body shape may be made, for example, by using information about the actual body shape of the user Ui or the like periodically acquired by the acquisition unit 12.

Further, in the body shape management system 1, when it is determined that the actual body shape of the user, who has received the message extracted by the extraction unit 14, has gotten closer to his/her desired body shape owing to that extracted message, the avatar adjustment unit 15 may increase, after this extracted message containing a predetermined content, the degree of change in the future body shape of the avatar of the user, who has received the message, that occurs in response to a subsequent message containing the predetermined content posted by the user, who has posted the extracted message. For example, when it is determined that the actual body shape of the user U1 has gotten closer to his/her desired body shape owing to a message containing a predetermined content posted for the user U1 by the user U2, the avatar adjustment unit 15 may determine that the contribution of the message from the user U2 is high and hence increase, after this message containing the predetermined content, the degree of change in the future body shape of the avatar of the user U1 that occurs in response to a subsequent message containing the predetermined content posted by the user U2. Further, in this case, the avatar output unit 13 may attach a mark indicating a contributing result by which the actual body shape of the user U1 has gotten closer to his/her desired body shape to the avatar of the user U2 or in the vicinity of the avatar. Alternatively, the avatar output unit 13 may change either the color or the shape of the avatar of the user U2 according to the contributing result by which the actual body shape of the user U1 has gotten closer to his/her desired body shape. In this way, it is possible to encourage the user U2 to post cheering messages.

### (Operation of Body Shape Management System 1)

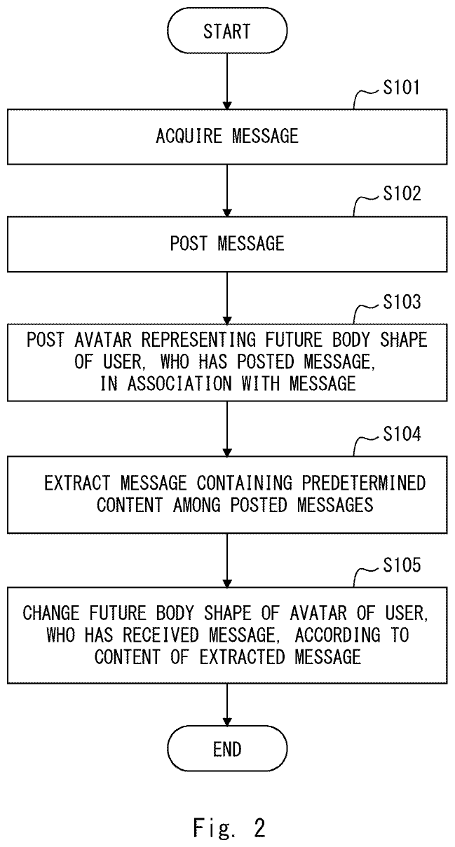

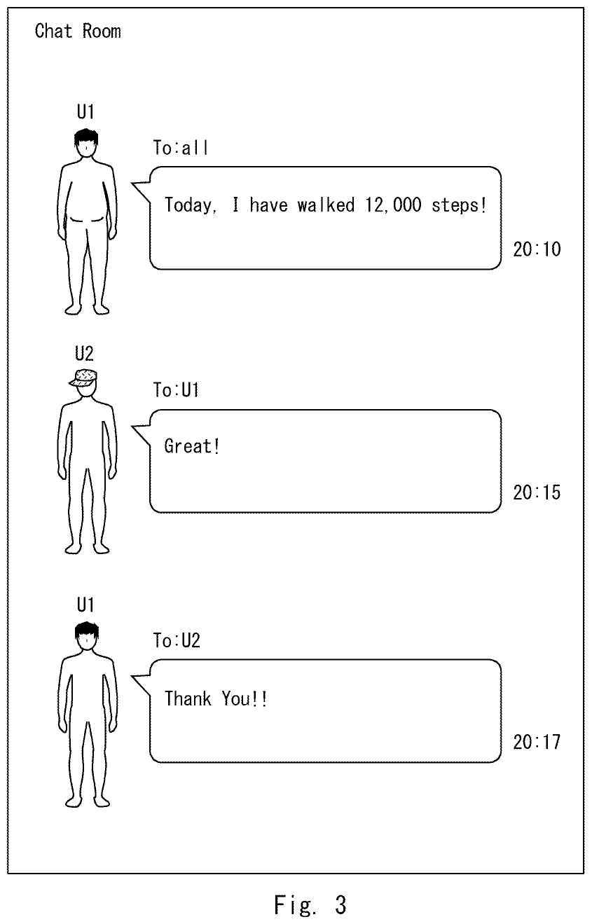

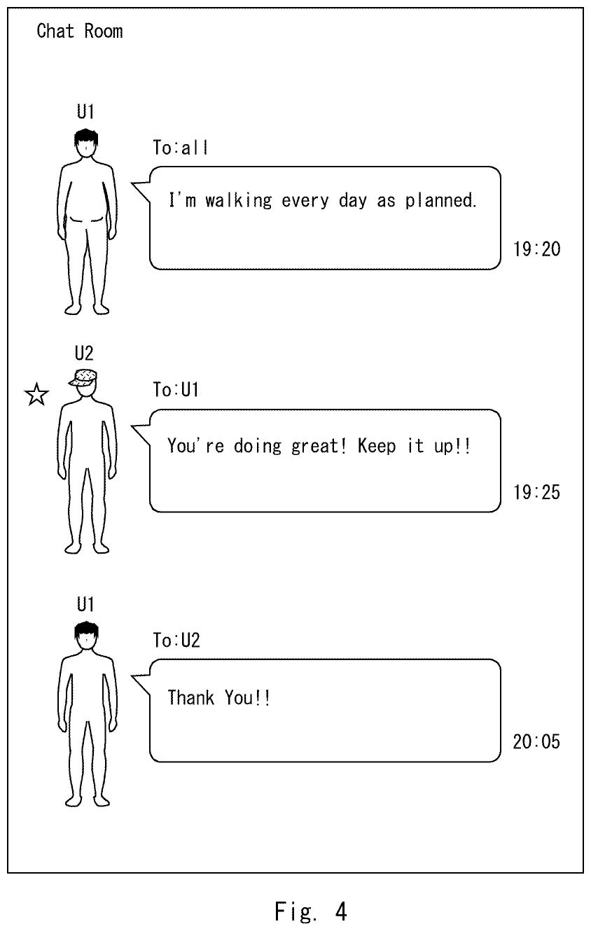

Next, operations performed by the body shape management system 1 will be described with reference to FIGS. 2 to 4. FIG. 2 is a flowchart showing operations performed by the body shape management system 1. FIGS. 3 and 4 show examples of chat rooms displayed on a monitor of a user terminal.

In the body shape management apparatus 10, the acquisition unit 12 acquires a message from a user Ui to be posted in the chat room, transmitted from an arbitrary user terminal 20_i (i is an integer from 1 to n) among the user terminals 20_1 to 20_n (Step S101). The message from the user Ui acquired by the acquisition unit 12 is posted in the chat room by the management unit 11 (Step S102). As shown in FIGS. 3 and 4, messages by users U1 and U2 are posted in the chat room.

After that, the avatar output unit 13 posts, in the chat room, an avatar representing a future body shape, i.e., a body shape expected after a lapse of a set period, of the user Ui who has posted a message in the chat room in association with the message posted by the user Ui (Step S103). As shown in FIGS. 3 and 4, avatars representing future body shapes, i.e., body shapes expected after a lapse of a set period, of the users U1 and U2 are posted in the chat room in association with the messages posted by the users U1 and U2, respectively.

After that, the extraction unit 14 extracts a message containing a predetermined content from among messages posted in the chat room (Step S104). Note that in the case of messages posted for the user U1 by the user U2, the message containing the predetermined content includes one of a message containing a content complimenting an effort of the user U1 to achieve the desired body shape, a message containing a content complimenting the user U1 that his/her actual body shape has gotten closer to his/her desired body shape, and a message containing a content cheering the user U1 up so that his/her actual body shape gets closer to his/her desired body shape. As shown in FIG. 3, the extraction unit 14 extracts a message containing “Great!” posted for the user U1 by the user U2. As shown in FIG. 4, the extraction unit 14 extracts a message containing “You're doing great!” posted for the user U1 by the user U2.

After that, when a message containing a predetermined content is extracted by the extraction unit 14, the avatar adjustment unit 15 changes the future body shape of the avatar of the user Ui, who has received the message, so that, for example, the body shape of the avatar gets closer to the desired body shape (Step S105). As shown in FIG. 3, since the message containing “Great!” is extracted by the extraction unit 14, the avatar adjustment unit 15 has changed the future body shape of the avatar of the user U1, who has received this message, so that it gets closer to the desired body shape (to make the body shape leaner). As shown in FIG. 4, since the message containing “You're doing great!” is extracted by the extraction unit 14, the avatar adjustment unit 15 has changed the future body shape of the avatar of the user U1, who has received this message, so that it gets closer to the desired body shape (to make the body shape leaner). Note that in the example shown in FIG. 4, since it was determined in the past that the actual body shape of the user U1 had gotten closer to his/her desired body shape owing to a message for the user U1 by the user U2, a star-shaped mark representing this contributing result is attached to the avatar of the user U2 (i.e., in the vicinity of the avatar).

Each of the user terminals 20_1 to 20_n can acquire (receive) information in the chat room and display the acquired information on a monitor by accessing the chat room through the network 50 through an operation performed by a respective one of the users U1 to Un. For example, as the user U1 watches that the future body shape of his/her avatar in the chat room displayed on the monitor of the user terminal changes, the user U1 becomes aware that he/she is being cheered up by other users and/or receives attention from other users, so that it is possible to encourage the user U1 to improve his/her body shape.

As described above, the body shape management system 1 according to this embodiment changes the future body shape of an avatar of a user that is posted together with a message from that user in the chat room in response to an encouraging message from other users in such a manner that, for example, the future body shape of the avatar gets closer to the desired body shape of the user. In this way, the body shape management system 1 can make the user be aware that, for example, he/she is being cheered up by other users, and thereby can encourage the user to improve his/her body shape.

Note that the present disclosure is not limited to the above-described embodiments, and they can be modified as appropriate without departing from the scope and spirit of the disclosure.

Further, in the present disclosure, some or all of the control processes performed in the body shape management system can be implemented by having a CPU (Central Processing Unit) execute a computer program.

In the aforementioned program includes instructions (or software codes) that, when loaded into a computer, cause the computer to perform one or more of the functions described in the embodiments. The program may be stored in a non-transitory computer readable medium or a tangible storage medium. By way of example, and not a limitation, the non-transitory computer readable media or tangible storage media can include a RAM (Random-Access Memory), a ROM (Read-Only Memory), a flash memory, an SSD (Solid-State Drive) or other types of memory technologies, a CD-ROM, a DVD (Digital Versatile Disc), a Blu-ray (Registered Trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A body shape management system comprising:
an avatar output unit configured to post an avatar representing a future body shape of a first user in a chat room in association with a message posted by the first user in the chat room, the future body shape of the first user being his/her body shape expected after a lapse of a set period;
an extraction unit configured to extract a message containing a predetermined content from a message posted for the first user by a second user different from the first user; and
an avatar adjustment unit configured to change the future body shape of the avatar of the first user according to the content of the message extracted by the extraction unit.

2. The body shape management system according to claim 1, wherein when one of a message containing a content complimenting an effort of the first user to achieve the desired body shape, a message containing a content complimenting the first user that his/her body shape has gotten closer to his/her desired body shape, and a message containing a content cheering the first user up so that his/her body shape gets closer to his/her desired body shape is extracted from among messages containing the predetermined content posted for the first user by the extraction unit, the avatar adjustment unit changes the future body shape of the avatar of the first user so that it gets closer to the desired body shape.

3. The body shape management system according to claim 1, further comprising an acquisition unit configured to acquire information about an actual body shape of the first user, wherein
the avatar output unit posts an avatar representing a future body shape corresponding to the actual body shape of the first user in the chat room.

4. The body shape management system according to claim 1, further comprising a predicted body shape calculation unit configured to calculate a predicted body shape of the first user according to at least a schedule of the first user stored in a database, the predicted body shape of the first user being a body shape that it is predicted the first user will have after a lapse of a set period, wherein
the avatar output unit posts an avatar representing a future body shape corresponding to the predicted body shape of the first user calculated by the predicted body shape calculation unit in the chat room.

5. The body shape management system according to claim 1, further comprising a notification unit configured to, when it is determined that the actual body shape of the first user has gotten closer to his/her desired body shape owing to the message extracted by the extraction unit, notify the first user that his/her body shape has gotten closer to his/her desired body shape.

6. The body shape management system according to claim 5, wherein when it is determined that the actual body shape of the first user has gotten closer to his/her desired body shape owing to the message extracted by the extraction unit, the notification unit posts a message indicating that his/her body shape has gotten closer to his/her desired body shape in the chat room.

7. The body shape management system according to claim 1, wherein when it is determined that the actual body shape of the first user has gotten closer to his/her desired body shape owing to the message extracted by the extraction unit, the avatar adjustment unit increases, after this extracted message containing the predetermined content, a degree of change in the future body shape of the avatar of the first user that occurs in response to a subsequent message containing the predetermined content posted by the second user, who has posted the extracted message.

8. The body shape management system according to claim 1, wherein when it is determined that the actual body shape of the first user has gotten closer to his/her desired body shape owing to the message extracted by the extraction unit, the avatar output unit attaches a mark to an avatar of the second user, who has posted the extracted message, or in a vicinity of the avatar of the second user, the mark indicating a contributing result by which the actual body shape of the first user has gotten closer to his/her desired body shape.

9. The body shape management system according to claim 1, wherein the avatar output unit changes at least one of a color and a shape of the avatar of the second user, who has posted the message extracted by the extraction unit, according to the contributing result by which the actual body shape of the first user has gotten closer to his/her desired body shape.

10. The body shape management system according to claim 1, wherein the set period is changeable.

11. A method for controlling a body shape management system according to the present disclosure comprising:
posting an avatar representing a future body shape of a first user in a chat room in association with a message posted by the first user in the chat room, the future body shape of the first user being his/her body shape expected after a lapse of a set period;
extracting a message containing a predetermined content from a message posted for the first user by a second user different from the first user; and
changing the future body shape of the avatar of the first user according to the content of the extracted message.

12. A non-transitory computer readable storage medium storing a control program for causing a computer to perform:
a process for posting an avatar representing a future body shape of a first user in a chat room in association with a message posted by the first user in the chat room, the future body shape of the first user being his/her body shape expected after a lapse of a set period;
a process for extracting a message containing a predetermined content from a message posted for the first user by a second user different from the first user; and
a process for changing the future body shape of the avatar of the first user according to the content of the extracted message.
